(19) United States
(12) Patent Application Publication
(10) Pub. No.: US 2003/0152606 A1
(43) Pub. Date: Aug. 14, 2003

(54) Inorganic resorbable bone substitute material and production method

(76) Inventor: Thomas Gerber, Papendorf (DE)

Correspondence Address: JORDAN AND HAMBURG LLP, 122 EAST 42ND STREET, SUITE 4000, NEW YORK, NY, 10168, US

(21) Appl. No.: 10/169,424

(22) PCT Filed: Jan. 25, 2001
(86) PCT No.: PCT/EP01/00803

(30) Foreign Application Priority Data
Jan. 28, 2000 (DE) 10003824.7
Dec. 2, 2000 (DE) 10060036.0

(51) Int. Cl. A61F 2/00

(52) U.S. Cl. 424/426

(57) ABSTRACT

The invention relates to an inorganic resorbable bone substitute material based on calcium phosphates and to a method for producing the same. The material is characterized in that it comprises a loose cyrstal structure, i.e., the crystallites are not tightly connected as in a solid body (ceramic), but they are interconnected via only a few molecular groups. The volume which is occupied by collagen in natural bone is provided in the material as interconnecting pores in the nanometer range. A second pore size, also interconnecting and in the range of a few micrometers, permits collagen fibers to grow inside during tissue formation. These fibers are nucleators for the inserting biomineralization (formation of the endogenous biological apatite). The material contains a third interconnecting pore category which is modeled on the spongiosa and thus ranges from approximately 100 μm to 1000 μm while enablign a vscularization of blood vessels such that the resorption and the bone regeneration not only occurs as the surface of healthy bone but also takes place throughout the entire defect. The high inner surface of the material permits the bonding of endogenous or synthetic growth factors. The invention also realtes to a method for producing such a material which is characterized in that a highly viscous suspension of a sol of one or more oxides of the elemtns X (X=Al, Ca, Mg, P, Si, Ti, Zr) tht is mixed with a crystalline powder is forced through a nozzle or a nozzle system and subsequently formed into any desired shape so that an open porous structure with a size corresponding tot hat of the filament diameters results by packing the fibers from the highly viscous suspension whose viscosity prevents the material from dispersing.

The invention relates to an inorganic resorbable bone substitute material based on calcium phosphates. Bone transplantation is, after administration of blood constituents, the second commonest form of transplantation in humans (Fox, R.: New bone. The Lancet 339, 463f. (1992)). Thus, in the USA in 1993, 250 000 bone transplantations were performed (Kenley et al.: Biotechnology and bone graft substitutes. Pharmaceut. Res. 10, 1393 (1993)). The replacement of bone defects which are post-traumatic, occur as a consequence of osteomyelitis and tumor operations, or are osteoporotic involves major clinical importance because this is the only possibility for functionally comprehensive rehabilitation.

The method referred to as “gold standard”, of removal of autologous bone, usually from the hip crest [sic], entails additional costs, risks and stress for the patient and there are limits on the amount of bone available. The removal defects, which are in some cases extensive, are often painful for a long time and there is an increased risk of infection. To avert these problems, various alloplastic and allogeneic materials have been developed, but none of them has shown clinically satisfactory results to date (Reuter, F., Kübler, N. R.: Die Wiederherstellung des Unterkiefers. Dtsch. Ärzteblatt 96 A, 1054ff. (1996)). Methods to date for filling or regeneration of defects (bank material, plastics, inorganic materials) have disadvantages and risks such as viral infection, fibrous reaction of surroundings, avitality or lack of resorption.

The development of an innovative group of inorganic biomaterials as alternative to autologous osteoplasty represents a considerable advance because a secondary operation with its increased costs, risks and complications can be avoided, and the disadvantages of other methods, such as, for example, the transmission of diseases (HIV, hepatitis, encephalitis, inter alia) or serious immune responses to the implant, do not apply in principle. A significant gain in quality for the persons affected results if the incorporation phase until load-bearing is possible is shortened.

Regeneration of bone tissue can take place in three different ways: osteogenesis, osteoinduction and osteoconduction (Kübler, N. R.: Osteoinduktion und -reparation. Mund Kiefer Gesichts Chir. 1, 2ff. (1997)). Osteoconduction means growth, originating from bone tissue which is present along a conducting structure, whereas stimulation of the differentiation of bearing tissue cells to osteoblasts is referred to as osteoinduction. Osteogenesis by contrast represents formation of new bone from vital transplanted bone cells.

The essential requirement for a bone substitute material is resorbability. Bone is continuously passing through a phase of formation and breakdown, called remodeling. A bone substitute material should take part in this remodeling and thus be replaced by natural bone within a certain time (about 12 months, depending on the size of the defect). Natural bone is broken down by osteoclasts. With an ideal bone substitute, resorption should also be effected by osteoclasts because breakdown of the material is coupled to the formation of new bone in this way. All other resorption mechanisms proceed in the final analysis via resorptive inflammation which—especially if it becomes too severe—always inhibits formation of new tissue.

Bone is a “composite material” composed of an inorganic mineral portion and an organic portion (collagen). The mineral is biogenic hydroxyapatite (HA), a calcium phosphate. Pure HA has the structural formula Ca₁₀(PO₄)₆(OH)₂. By contrast, biogenic HA has some substitutions. Thus, there is substitution of Mg, F and Cl (<1% by weight) for Ca, and CO₃ groups for PO₄ groups (5.8% by weight in bone) (E. M. Carlisle: A possible factor in bone calcification, Science 167, pp. 279-280 (1970)). The crystal structure of the minerals is hexagonal with the lattice parameters substantially corresponding to those of synthetic HA (differences in the 3rd decimal, Angstrom range). The minerals arranged between the collagen fibers have a pronounced platelet shape. The average dimensions are 45 nm×30 nm×3 nm. Electron microscopic investigations demonstrate that single crystals with structural defects are involved (E. M. Carlisle: In vivo requirement for silicon inarticular cartilage and connective tissue formation in the chick, J. Nutr. 106, pp. 478-484 (1976)), probably caused by the substitutions mentioned. The microstructure of the collagen/mineral composite can briefly be described as follows. Collagen fibrils arrange themselves into parallel bundles in accordance with the external stress. These are mechanically strengthened by HA crystals arranged between the fibrils. The platelets moreover lie flat on the fibrils, with the crystallographic c axis of the minerals being oriented parallel to the long axis of the fibrils. The site of attachment to the collagen fibers is determined by the hierarchical structure of collagen (molecule—procollagen (tipel [sic] helix)—microfibril). Procollagen molecules assemble themselves in parallel with a characteristic displacement. In the longitudinal direction there are 35 nm gaps between the procollagen molecules. The eventual result is a structure with a 64 nm period (Parry, D. A.: The molecular and fibrillar structure of collagen and its relationship to the mechanical properties of connective tissue. Biophys. Chem. 1988 February; 29(1-2):195-209. Review). From this basic structure there is formation, through oriented assemblage of the fibrils, of more or less complicated superstructures (tendons, lamellated bone, woven bone; structural models see (Arsenault, A. L.: Crystalcollagen relationships in calcified turkey leg tendons visualized by selected-area dark field electron microscopy. Calcif. Tissue Int. 1988 October; 43(4):202-12), (Traub, W.; Arad, T.; Weiner, S.: Origin of mineral crystal growth in collagen fibrils. Matrix. 1992 August; 12(4):251-5) and (Landis, W. J.; Hodgens, K. J.; Song, M. J.; Arena, J.; Kiyonaga, S.; Marko, M.; Owen, C., McEwen, B. F.: Mineralization of collagen may occur on fibril surfaces: evidence from conventional and high-voltage electron microscopy and three-dimensional imaging. J. Struct. Biol. 1996 July-August; 117(1):24-35)). The gap between the procollagen molecules is regarded as the site of primary nucleation [sic].

It is ideal for a bone substitute material that it has a pore structure like that present in spongiosa. In other words, interconnecting pores with a diameter of about 0.2 mm to 0.8 mm must exist. This makes it possible for blood vessels to grow into the material, and thus the remodeling process is in fact made possible.

Porous bioceramics composed of tricalcium phosphate (TCP)/hydroxyapatite (HA) and TCP/monocalcium phosphate monohydrate (MCPM) are the subject of international animal experimental research, both isolated and in combination with BMP and bone marrow cells for osteoconduction and osteoinduction (Wippermann, B. et al.: The influence of hydroxyapatite granules on the healing of a segmental defect filled with autologous bone marrow. Ann. Chir. Gynaecol. 88, 194ff. (1999); Anselme, K. et al.: Associations of porous hydroxy-apatite and bone marrow cells for bone regeneration. Bone 25 (Suppl. 2), 51Sff. (1999); Niedhart, C. et al.: BMP-2 in injizierbarem Tricalciumphosphat-carrier ist in Rattenmodell der autologen Spongiosaplastik biomechanisch uberlegen. Z. Orthop. 137 (Suppl. I), VI-283 (1999); Penel, G. et al.: Raman microspectrometry studies of brushite cement: in vivo evolution in a sheep model. Bone 25 (Suppl. 2), 81Sff. (1999); Brown, G. D. et al.: Hydroxyapatite cement implant for regeneration of periodontal osseous defects in humans. J. Periodontol. 69(2), 146ff. (1998); Flautre, B. et al.: Volume effect on biological properties of a calcium phosphate hydraulic cement: experimental study in sheep. Bone 25 (Suppl. 2), 35Sff. (1999)). The open-pore lattice-like structure of resorbable TCP/HA promotes regenerate formation (Jansson, V. et al.: Knochen-/Knorpel-Regeneration in Bioimplantaten-Ergebnisse einer tierexperimentellen Studie. Z. Orthop. 137 (Suppl. I), VI-307 (1999)). There is evidence that integration and regeneration in the case of macroporous HA ceramics proceeds by resorption, microfracture and renewed osteoconduction (Boyde, A. et al.: Osteo-conduction in a large macroporous hydroxyapatite ceramic implant: evidence for a complementary integration and disintegration mechanism. Bone 24, 579ff. (1999)). It would be possible to achieve a further increase in the regeneration potential by combination with BMP (bone morphogenic protein (Meraw, S. J. et al.: Treatment of peri-implant defects with combination growth factor cement. J Periodontol 71(1), 8ff. (2000) or osteoprogenitor cells through additional osteoinduction.

A composite material composed of organic and inorganic materials proves to be unfavorable as bone substitute because exogenous organic constituents cause rejection reactions by the body (immune responses) or lead to unwanted resorptive inflammations.

A large number of porous ceramics [lacuna] described as bone substitute in the patent literature. In U.S. Pat. No. 5,133,756; 1992 the ceramic is produced from the spongiosa of cattle bones and thus has the required pore structure. The entire organic matrix is removed and the ceramic portion is heat treated at temperatures of from 1100° C. to 1500° C. Another method (U.S. Pat. No. 4,861,733; 1989) starts from the framework of natural corals and converts the calcium carbonate in a hydrothermal process into calcium phosphate. The advantage of this method is that the pore structure (size distribution, morphology) is ideal for bone tissue to grow into.

The critical disadvantage of these ceramics is that they are not resorbable. The significance of this for the described materials is that there is indeed excellent growth into the pore structure by the bone tissue. However, the fixed crystal structure of the ceramic is not involved in the bone remodeling. It therefore remains a foreign body and influences the mechanical properties. Inflammations occur at the junction of tissue and ceramic in particular during bone growth.

Resorbable ceramics based on tricalcium phosphate are described (U.S. Pat. No. 5141511, 1992). A fixed crystal structure produced by sintering processes is involved in this case too. Pores are introduced into the material only in the order of magnitude of the spongiosa. Resorption takes place on the basis of the solubility of the tricalcium phosphate. This leads to a local increase in the ion concentration, and resorptive inflammation occurs.

Bioactive glasses are likewise offered as bone substitute material (U.S. Pat. Nos. 6,054,400, 200; 5,658,332, 1997). The inorganic material is in these cases in the form of a glassy solid. Pores in the order of magnitude of spongiosa permit ingrowth of tissue. Smaller pores are not present in the material.

Glass ceramics are also offered as bone substitutes (U.S. Pat. No. 5,998,1412 [sic], 1999). They are comparable with bioactive glasses, with the calcium phosphate being present as crystalline component in a glass matrix. A further group of substances developed for use as bone substitute are calcium phosphate cements (U.S. Pat. Nos. 5,997,624, 1999; 5,525,148, 1996). The critical disadvantage of this group of substances is that no defined interconnecting pores are introduced into the material, which means that they are confined to very small bone defects.

The present invention is by contrast based on the object of providing a bone substitute material which assists the formation of bone tissue (which is thus osteoconductive or osteoinductive) and which is resorbed via the natural processes of bone remodeling. It is further intended to indicate a method for producing such a bone substitute material.

The object is achieved according to the invention by a material having the features of claim 1. The material has a loose crystal structure of calcium phosphates, i.e. the crystallites are not tightly joined together as in a solid (ceramic) but are connected together only via a few molecular groups. The volume occupied in natural bone by collagen is present in the material as interconnecting pores in the nanometer range. A second pore size, likewise interconnecting and in the region of a few micrometers, makes it possible for collagen fibers to grow in during tissue formation. These fibers form nuclei for the onset of biomineralization (formation of endogenous biological apatite). The material comprises a third interconnecting pore category which simulates the spongiosa and is thus in the range from 100 μm to 1000 μm and thus makes ingrowth of blood vessels possible, whereby the resorption and the formation of new bone not only takes place as front starting from healthy bone but also outward from the entire defect.

The pore structure means that the -developed material is outstandingly suitable for taking up endogenous (e.g. bone marrow fluid) or exogenous (e.g. BMPs) osteoinductive components. This achieves extreme tissue compatibility and thus rapid ingrowth of bone tissue. The loose crystal structure makes resorption through osteoclasts possible.

The calcium phosphate primarily used is a hydroxyapatite which matches biological apatite in size of crystallite. A second soluble calcium phosphate component (β-tricalcium phosphate or bruschite [sic]) may be chosen as local calcium phosphate supplier for the biomineralization starting on the collagen fibers. The soluble components are to be present in the concentration that only slight or no resorptive inflammation occurs, which is not to prevent formation of new tissue.

There are increasing reports in the literature of the beneficial effect of SiO₂ on collagen and bone formation.

The results are obtained both with in vitro and with in vivo experiments.

Carlisle (E. M. Carlisle: A possible factor in bone calcification, Science 167, pp. 279-280 (1970)) reports that silicon is an important trace element in the formation and mineralization of bone. A silicon deficiency in animal experiments on chickens and rats produces a defective bone structure (E. M. Carlisle: In vivo requirement for silicon inarticular cartilage and connective tissue formation in the chick, J. Nutr. 106, pp. 478-484 (1976)). The silicon is used by various authors in different forms in the experiments. Thus, Keeting et al. (P. E. Keeting et al: Zeolite a increases proliferation, differentiation, and transforming growth factor β production in normal adult human osteoblast-like cells in vitro, J. of bone and mineral research, Vol.7, No.11, pp. 1281-1289 (1992)) use silicon-containing zeolites A for their experiments and find a beneficial effect on cell growth and cell division of cultivated cells of a human cell line. It is, of course, important in this connection that other elements such as, for example, aluminum with an adverse effect also enter the system thereby.

The effect of silicon on bone formation is investigated on cell lines in vitro by Reffitt et al. (D. Reffitt et al.: Silicon stimulated collagen type I synthesis in human osteoblast-like cells, Bone 23(5), p. 419 (1998)). Stimulation of type I collagen synthesis is found. The loss of bone mass by osteoporotic rats was investigated in an animal experiment (H. Rico et al.: Effect of silicon supplement on osteopenia induced by ovariectomy in rats, Calcif. Tissue Int. 66(1), pp. 53ff. (2000)). It was found in this case that rats receiving 500 mg of Si per kg of feed showed no loss of bone mass, in contrast to the animals which had no Si in the feed. Lyu (K. Lyu, D. Nathason, L. Chou: Induced osteogenesity [sic] in vitro upon composition and concentration of silicon, calcium, and phosphorous [sic]. Sixth World Biomaterials Congress Transactions 2000, 1387) finds with in vitro experiments that Si plays an important part in osteogenesis, and there is a correlation between osteogenesis activity and Si concentration (from 10 to 100 ppm Si in culture medium).

The beneficial aspect of SiO₂ in bone formation is taken up by the described bone substitute material in that nanoporous SiO₂ is introduced into the loose crystal structure of the bone substitute material. Nanoporous SiO₂ is chosen in order, on the one hand, to achieve good solubility and, on the other hand, to ensure a large internal surface area.

One method for achieving the object on which the invention is based exhibits the measures of claim 6. They consist of a highly viscous suspension consisting of a sol of one or more oxides of the elements X (X=Al, Ca, Mg, P, Si, Ti, Zr) being mixed with a crystalline powder, forced through a nozzle or a nozzle system and subsequently introduced into any suitable mold so that the packing of the fibers from the highly viscous suspension, the viscosity of which prevents the material flowing out of control, results in an open pore structure in the size range of the diameters of the fibers, but the fibers are connected through the as yet incomplete gel transition at the points of contact. These open pores, whose size extends from 50 μm to a few 1000 μm, that is to say in a considerably larger range than the pores produced by the sol-gel process (Patent DE 198 25 419 A1), make rapid ingrowth of tissue and, in particular, of blood vessels possible. This ensures resorption of the material.

The highly viscous suspension is produced by mixing as homogeneously as possible calcium phosphate powder or granules, which can be varied through the component used, the particle size distribution, the morphology, the degree of crystallinity and lattice defects which are present, with a sol of one or more oxides of element X (X=Al, Ca, Mg, P, Si, Ti, Zr).

The mixture is then packed into a container so that no air is present in the closed container, and the container is rotated around a horizontal axis in order to prevent sedimentation of the heavier solid portions. The diameter of the nozzle or nozzles is preferably in the range from 50 μm to 1000 μm, while 200 μm achieves a value which corresponds to the diameter of the trabecula [sic] in bone and is technically easy to achieve.

The fibers resulting from the highly viscous suspension through the nozzles or the nozzle system are forced into the suitable mold, such as a cylinder, hollow cylinder or segment of a hollow sphere, in such a way that the pores determined by the packing of the fibers require a particular proportion by volume of, preferably 50%, and connection of the fibers which are in contact is ensured.

The viscosity of the suspension forming the fibers must not be so low that the fibers flow into one another.

It may be necessary where appropriate, especially if very thin fibers are to be produced, to increase the viscosity after passing through the nozzle or nozzles in order on the one hand to prevent blockage of the nozzles (lower viscosity), and on the other hand to avoid uncontrolled flow of the fibers (higher viscosity).

This is achieved by rapidly removing solvent from the suspension after leaving the nozzle(s). This can take place through a rapid increase in temperature and/or through a reduction in the partial pressure of the solvent. It proves simplest to flush the fiber packing with hot dry air.

In order to improve the strength of the highly porous shaped article, the packing of the fibers can be impregnated with a suspension of the same composition as the initial suspension, choosing for this a viscosity of the suspension which ensures that parts [sic] of the suspension remains suspended between the fibers and, with the gel formation, makes better linkage of the fibers possible and, at the same time, prevents blockage of the large interconnecting pores.

Drying of the shaped article is preceded by aging of the gel structure. A saturated solvent atmosphere prevents premature drying.

The drying is subsequently carried out at a temperature of, preferably, 90-150° C. for 2 hours. The gel then remains in a nanoporous state, which facilitates resorption. If the strength is to be increased, a thermal treatment in a range between 600° C. and 800° C. takes place.

After the drying, the highly porous shaped article is buffered, preferably with phosphate buffer at pH 7.2 The drying process which is necessary thereafter is associated with a sterilization.

The invention is explained below by means of examples. However, it is not restricted to these examples.

### EXAMPLE 1

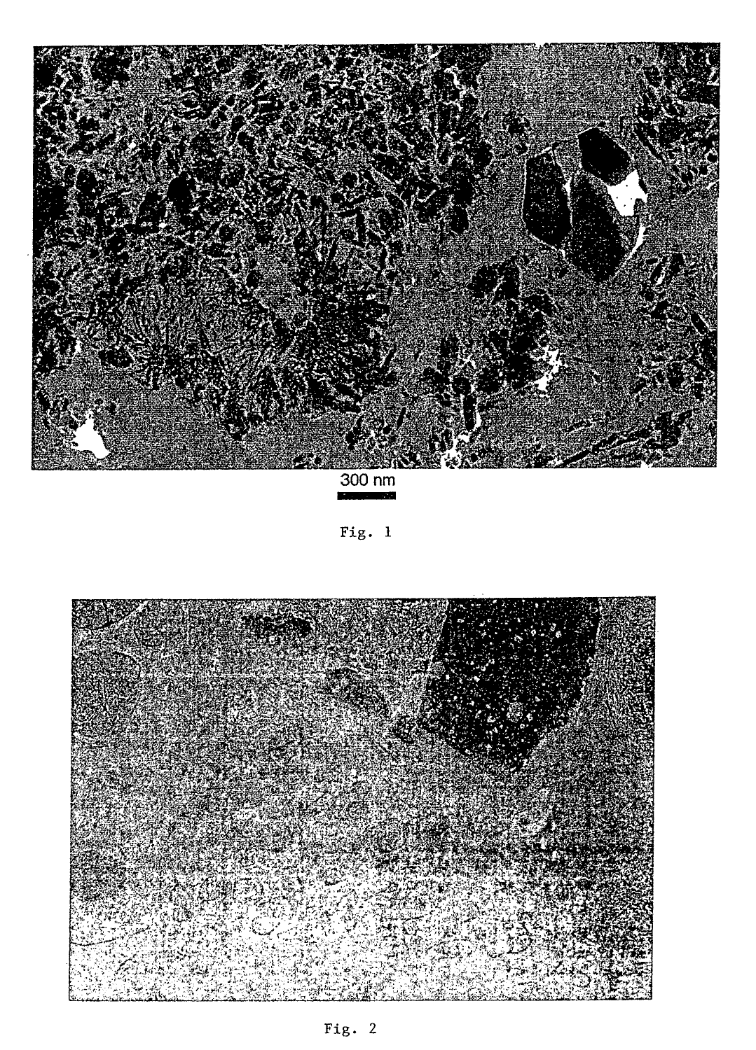

FIG. 1 shows a transmission electron micrographs [sic] of sections of the biomaterial embedded in epoxide. The smooth surfaces are the pores filled with epoxide. The loose crystal structure is clearly evident and can be influenced by different calcium phosphate powders of differing crystal morphology. A ratio of 60% hydroxyapatite (HA) and 40% β tricalcium phosphate (TCP) was chosen for the calcium phosphate for this example. The larger crystals in the figure are the β TCP portions.

The porosity has the order of magnitude of the crystallites. Thus, a large surface area exists and is wetted by body fluid in vivo.

The figure simultaneously demonstrates that marked interconnecting pores in the μm range exist (here are filled with epoxide due to the TEM preparation) and permit unhindered ingrowth of collagen fibers.

Gottinger minipigs were used for the animal experiments. The animals were adult (one year old) and had a weight between 25 and 30 kg. The bone defects exceeded the critical size of 5 cm³; their dimensions were about 3.0 cm×1.5 cm×1.5 cm. They were made in the lower jaw, completely filled with the bone substitute material and closed with periostum. After 5 weeks, the pigs were sacrificed, and the lower jaws were removed and X-ray, histological and scanning microscopic investigations were carried out. The animal experiments were evaluated after 5 weeks in order to study the initial stage of bone regeneration. Good ossification is detectable in the marginal zone. Histological sections from the marginal zone demonstrate very good bone formation. The biomaterial is partly covered by young bone (FIG. 2).

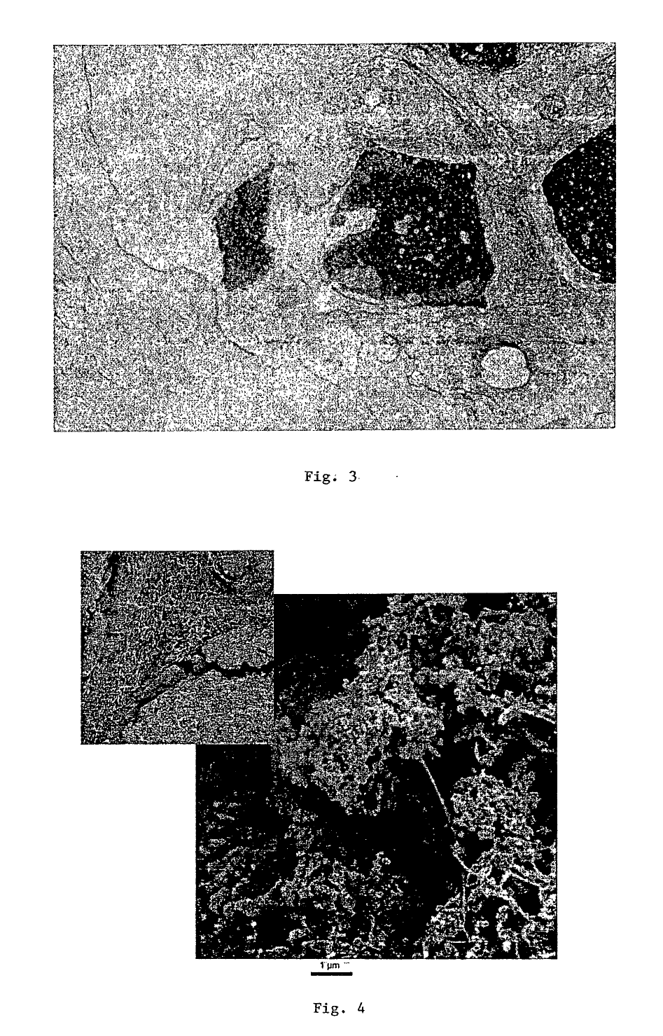

Clear signs of resorption are evident even after 5 weeks. The originally “round” material has acquired edges and corners and shows indentations typical of osteoclast activities (FIG. 3). It is moreover evident that the micrometer pores of the material are permeated by organic material. The SE micrographs confirm this impressively. FIG. 4 shows a scanning electron micrograph of a section from the middle of the defect and an enlarged detail. The micropores are permeated by collagen fibers, which in turn distinguish a mineralization, throughout the defect—also centrally where bone formation is not as advanced.

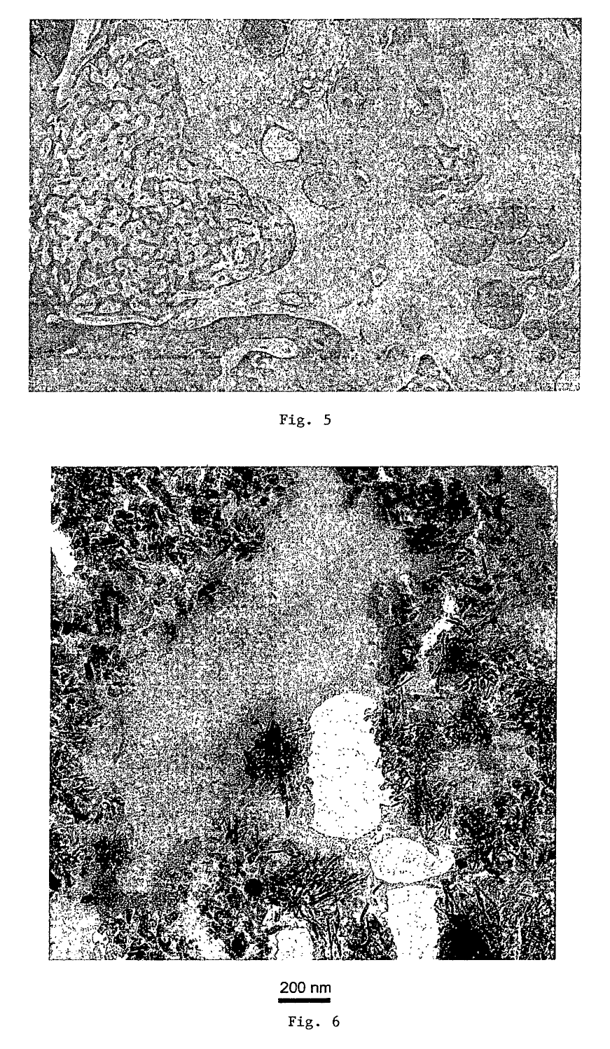

FIG. 5 shows a demineralized histological section (hemalum eosin). It is evident that the large pores of the biomaterial permit ingrowth of blood vessels starting from the margin.

### EXAMPLE 2

FIG. 6 shows a transmission electron micrographs [sic] of sections of the biomaterial embedded in epoxide. The smooth surfaces are again pores filled with epoxide. The loose crystal structure is clearly evident and differs from that of FIG. 1. Pure hydroxyapatite (HA) was used as calcium phosphate for this example.

The porosity has the order of magnitude of the crystallites. Thus, a large surface area exists and is wetted by body fluid in vivo.

The figure simultaneously demonstrates that marked interconnecting pores in the μm range exist (here filled with epoxide due to the TEM preparation) and permit unhindered ingrowth of collagen fibers.

### EXAMPLE 3

18 ml of water and 18 ml of hydrochloric acid standard solution are added with stirring to 60 ml of tetraethoxysilane.

After the hydrolysis, about 60 g of hydroxyapatite and 40 g of β tricalcium phosphate are added to this mixture. This suspension is rotated in a closed vessel, which is 100% filled, around a horizontal axis in order to prevent the phosphates being deposited on the bottom.

After 2 hours, the viscosity is so high that the sol is forced through a nozzle with a diameter of 1 mm, and stable fibers are produced and are brought to a rectangular shape as random packing that [sic] the fibers have about 50% of space.

The sample is then stored in a desiccator with saturated ethanol vapor for 12 h. Drying is then carried out in an oven at 120° C. for 2 h.

After the drying process, a pH of 7.2 is set using phosphate buffer.

The samples are dried in air, and later dried and sterilized at 200° C. (heating rate: 1° C./min; duration 3 hours).

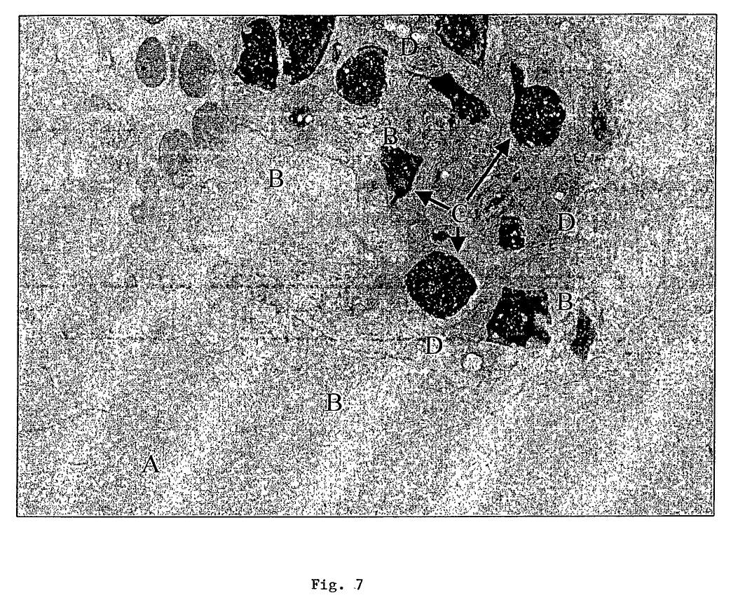

The animal experiments were carried out with Göttinger minipigs (fully grown, weighing about 60 kg). This entailed making a defect about 5 cm³ in the lower jaw and filling it with the material. After five weeks, the pigs were sacrificed in order to evaluate the initial stage of regeneration of the defect. A light micrograph of a histological section is shown in FIG. 7. Extremely rapid growth of bone (including blood vessels) into the pores of the bone substitute material and resorption of the material are evident. (A—bone of the lower jaw; B—newly formed bone; C—residues of the bone substitute material; D—blood vessels in the pores of the material). The originally thread-like structure has changed greatly due to resorption.

### EXAMPLE 4

18 ml of water and 18 ml of hydrochloric acid standard solution are added with stirring to 60 ml of tetraethoxysilane.

After the hydrolysis, about 40 g of hydroxyapatite are added to this mixture. This suspension is rotated in a closed vessel, which is 100% filled, around a horizontal axis in order to prevent the phosphates being deposited on the bottom.

After 2 hours, the viscosity is so high that the sol is forced through a nozzle with a diameter of 0.2 mm, and stable fibers are produced and are brought to a rectangular shape as random packing that [sic] the fibers have about 50% of space.

The sample is then stored in a desiccator with saturated ethanol vapor for 12 h. Drying is then carried out in an oven at 120° C. for 2 h.

1. An inorganic resorbable bone substitute material based on calcium phosphate, characterized:
a) in that a loose crystal structure of calcium phosphate with interconnecting pores in the nanometer range between the crystals is present, and the crystallites are connected only via a few molecular groups so that the proportions by volume which are occupied in natural bone by the collagen are now interconnecting pores,
b) in that the solids content of the bone substitute material is minimized and ingrowth of collagen fibers into the material is made possible since, in addition, interconnecting pores in the order of magnitude of from 1 μm to 10 μm permeate the material,
c) in that ingrowth of blood vessels is possible through further interconnecting pores which simulate those in spongiosa and are in the size range from 100 μm to 1000 μm.

2. An inorganic bone substitute material as claimed in claim 1, characterized in that the calcium phosphate is hydroxyapatite which preferably corresponds in size of crystallites to the biological apatite of bone.

3. An inorganic bone substitute material as claimed in claim 1, characterized in that it consists of hydroxyapatite and a soluble calcium phosphate which initiates, owing to the solubility, a rapid biomineralization of the collagen bundles which have grown into the micrometer pores (calcium and phosphorus supplier) and is present in the concentration which causes no resorptive inflammation inhibiting formation of new tissue.

4. An inorganic bone substitute material as claimed in claim 2 or 3, characterized in that nanoporous SiO₂ is incorporated into the loose crystal structure and is released on resorption of the material and thus speeds up collagen formation.

5. An inorganic bone substitute material as claimed in claim 1 or 4, characterized in that the large internal surface area is covered by synthetic or endogenous growth factors.

6. A method for producing an inorganic resorbable bone substitute material, characterized in that a highly visous suspension consisting of a sol of one or more oxides of the elements X (X=Al, Ca, Mg, P, Si, Ti, Zr) is mixed with a crystalline powder, forced through a nozzle or a nozzle system and subsequently introduced into any suitable mold so that the packing of the fibers from the highly viscous suspension, the viscosity of which prevents the material flowing out of control, results in an open pore structure in the size range of the diameters of the fibers.

7. A method as claimed in claim 6, characterized in that the viscosity of the suspension after leaving the nozzle or the nozzle system is increased and thus uncontrolled flow of the fibers in the mold is prevented.

8. A method as claimed in claim 6 and 7, characterized in that solvent is evaporated from the highly viscous suspension by a rapid increase in the temperature of the fibers after leaving the nozzle or the nozzle system, and the viscosity of the sol increases.

9. A method as claimed in claims 6 to 8, characterized in that solvent is evaporated by a rapid reduction in the partial pressure of the solvent in the suspension after leaving the nozzle or the nozzle system, and the viscosity of the suspension increases.

10. A method as claimed in claims 6 to 9, characterized in that the fibers are pressed into the mold in such a way that the pores produced through the packing of the fibers have the desired proportion of the volume of the molded article.

11. A method as claimed in claims 6 to 10, characterized in that the packing of the fibers is impregnated with a suspension of the same composition as the initial suspension, choosing for this a viscosity of the suspension which ensures that parts [sic] of the suspension remains suspended between the fibers and, with the gel formation, makes better linkage of the fibers possible and, at the same time, prevents blockage of the large interconnecting pores, with the viscosity being controlled via the gel formation time.

12. A method as claimed in claims 6 to 11, characterized in that the material is preferably dried in a temperature range from 90° C. to 200° C.

13. A method as claimed in claims 6 to 12, characterized in that the material is preferably thermally treated in a temperature range from 600° C. to 1000° C. to increase the strength.

14. A method as claimed in claims 6 to 13, characterized in that the material is preferably buffered with a phosphate buffer of pH 7.2.
